(19) United States
(12) Patent Application Publication
(10) Pub. No.: US 2003/0109837 A1
(43) Pub. Date: Jun. 12, 2003

(54) BRUSH TO CLEAR OCCLUDED STENTS

(76) Inventor: MARCIA MCBRIDE-SAKAL, BOLTON, MA (US)

Correspondence Address: FINNEGAN, HENDERSON, FARABOW, GARRETT & DUNNER, LLP, 1300 I STREET, NW, WASHINGTON, DC, 20005, US

(21) Appl. No.: 09/041,972

(22) Filed: Mar. 13, 1998

(51) Int. Cl. A61M 25/00

(52) U.S. Cl. 604/267; 606/159

(57) ABSTRACT

A cleaning brush and method are described for removing occluding material from an implanted stent. The device comprises a rotating brush mounted on the distal end of a catheter which can be inserted into the working lumen of a medical scope such as an endoscope or laparoscope. Irrigation and waste evacuation channels can also be provided in the device, and a guidewire is provided to facilitate accurate positioning.

### BACKGROUND OF THE INVENTION

1. Field of the Invention

The present invention is directed to a cleaning instrument for medical devices. More particularly, the invention is directed to a cleaning brush that can be employed with a medical scoping device, such as an endoscope, for mechanically clearing implanted stents that have become occluded by biological material such as biofilm and/or biliary sludge.

2. Background of the Related Art

Stenting has become the preferred method of palliation for disorders that obstruct lumens in the body. For example, stents are used to expand obstructive malignancies in the biliary, pulmonary, gastrointestinal, and urinary systems. They are also used to bridge and support anastomoses. These prostheses are implanted using minimally invasive techniques that significantly decrease morbidity and mortality. However, these procedures are not without complications, as stents frequently become occluded by biological matter.

For example, a significant percentage of implanted biliary stents become occluded by bacterial biofilm and/or biliary sludge within several months. The binary tract does not normally harbor microorganisms, as the daily flow of 800-1000 cc of bile and the continuous secretion of mucus through the biliary tract usually prevents the adherence of bacteria to the biliary mucosa. In addition, the sphincter of Oddi acts as a mechanical barrier to any gastric reflux from the duodenum, restricting the entrance of intestinal bacteria. However, when a stent is implanted, this barrier is breached and the biliary tract becomes contaminated.

Many bacteria that invade the biliary tract produce a dense polysaccharide capsule that shields the organism from host defenses and antibiotics. These bacteria adhere to stents and colonize within this protective matrix, forming a biofilm. Unfortunately, treatment with systemic antibiotics cannot eradicate these encapsulated colonies. The concentration of antibiotic secreted in bile significantly decreases with binary obstruction and most systemically delivered antibiotics are not even detectable in a completely occluded biliary system. Moreover, bacteria supported within a biofilm are resistant to many times the concentration of antibiotic that is lethal for the same bacteria in their planktonic state.

Once a biofilm is established, biliary sludge and cellular debris accumulate and eventually block the stent. This blockage must be mechanically cleared to prevent a recurrence of the jaundice and cholangitis that may have originally necessitated the stenting. Currently, the only mechanical means of clearing an occluded metallic stent is to insert a smaller diameter polyurethane stent through the blockage, or balloon trawl to remove debris from the lumen of the blocked stent.

Stents are typically constructed out of solid plastic, metallic mesh, or plastic-covered metal, and all have inherent advantages and disadvantages. Most physicians prefer plastic stents because they are removable and are considerably less expensive than metallic stents. However, plastic stents provide an ideal substrate for biofilm growth and these devices rapidly occlude.

Design modifications, such as increasing the size of the stent lumen, have been utilized to help extend patency rates. There is a direct correlation between the diameter of the lumen and the time it takes to occlude. However, the size of the stent is ultimately limited to that of the endoscope channel and/or the ductal lumen. The overall lack of success with structural changes has led to alternative strategies, such as antimicrobial coatings that prevent bacterial colonization.

The rapid occlusion of plastic stents has also prompted the development of self-expanding metallic stents. These prostheses are easier to insert than their plastic counterparts, create minimal trauma during implantation, and show less tendency to migrate. In addition, metal stents are generally more resistant to biofilm buildup, as they have a wider lumen and a mesh design that provides less surface area for bacteria to colonize. Nevertheless, metal stents are susceptible to tumor ingrowth and overgrowth, which also leads to stent blockage.

In order to block tumor ingrowth, polyurethane-covered metallic stents have been developed. Unfortunately, even these stents may eventually occlude, because the plastic coating increases surface area and accelerates bacterial colonization. Covered stents also block mucociliary action in the tracheobronchial tree, causing mucous plugs. Furthermore, both covered and uncovered stents can be blocked by food if implanted in the esophagus or feces in the colon.

Occluded stents must be exchanged or cleared to prevent serious medical complications. Plastic stents can be replaced easily if they become blocked, but metallic stents LAW OFFICES are rapidly epithelialized and cannot be removed. Covered metallic stents are sometimes removable, but replacement is extremely costly and can cause significant trauma to the patient. The risk of reintervention is further increased because patients who require metallic stenting are usually elderly and in an advanced stage of disease.

Accordingly, there is a need for an effective method and device for clearing blocked stents. Such a method and device should extend the life of the prosthesis, alleviate complications associated with restenosis, and render the biofilm more susceptible to systemic antibiotic therapy.

### SUMMARY OF THE INVENTION

To achieve these advantages and in accordance with the purpose of the invention, as embodied and broadly described, the invention comprises a medical cleaning device having a rotatable brush mounted on the distal end of a rotating shaft disposed within a flexible elongated sheath which can be inserted into the lumen of an endoscope or similar surgical device for cleaning implanted stents.

In a further aspect of the invention, the rotatable shaft is hollow and defines a flow path that is in fluid communication with the interior of the brush. The brush has a plurality of apertures for dispensing a fluid for irrigation of the stent site.

In yet a further aspect of the invention, the space between the rotatable shaft and the outer sheath defines an elongated channel which terminates in apertures disposed in the distal end of the sheath. At the proxiral end of the channel, a vacuum source is attached to the sheath for applying suction through the channel to the end of the sheath proximate the rotating brush to facilitate removal of debris cleaned from the stent.

In still a further aspect of the invention, a narrow elongated second channel extends concentrically through the rotatable shaft to accommodate a guide wire which is passed through the channel and the end of the brush to assist in positioning the brush in the area of the stent.

In another aspect of the invention, the rotatable shaft is concentrically disposed within a non-rotating elongated hollow shaft disposed within the sheath. The sheath can also include one or more additional non-rotatable elongated hollow tubular members extending parallel to the rotatable shaft within the elongated sheath. At least one of the additional hollow tubular members can be connected to a vacuum source and have a distal end proximate the brush to facilitate removal of debris from the stent site.

In yet another aspect of the invention, a method is provided for carrying out the clearing of an implanted occluded stent by means of a rotating brush introduced into the stent, for example, through the working lumen of an endoscope with irrigation and vacuum removal of abraded debris.

In still another aspect of the invention, a method is provided for clearing an implanted stent and treating the site to inhibit future occlusion.

Additional features and advantages of the invention will be set forth in the description which follows, and in part will be apparent from the description or may be learned by practice of the invention. The objectives and other advantages of the invention will be realized and attained by the apparatus particularly pointed out in the written description and claims hereof as well as the appended drawings.

It is to be understood that the foregoing general description and the following detailed description are exemplary and explanatory and are intended to provide further explanation of the invention as claimed.

### BRIEF DESCRIPTION OF THE DRAWINGS

Reference will now be made in detail to the present preferred embodiments of the invention illustrated in the accompanying drawings. Wherever possible, the same reference numbers are used in the drawings and the description to refer to the same or like parts.

The accompanying drawings are included to provide a further understanding of the invention and are incorporated in and constitute a part of this specification. The drawings illustrate several embodiments of the invention and together with the description serve to explain the principles of the invention.

FIG. 1 is a side perspective view illustrating an embodiment of the invention wherein both a rotating brush and separate vacuum canula are provided in the surgical cleaning device.

FIG. 2 is a side perspective view illustrating an additional embodiment of the invention wherein the rotating brush of the invention is provided with a fluid irrigation channel as well as provision for vacuum evacuation of the site being cleansed.

FIG. 3 is a side perspective view illustrating an alternative embodiment wherein a vacuum channel is concentric with the rotating shaft of the brush.

FIG. 4 is a side perspective view illustrating yet a further embodiment of the device providing for irrigation of the stent site.

FIG. 5 is a side perspective view illustrating retraction of the brush head in accordance with the invention.

FIG. 6 is a side perspective view illustrating the mechanism for retraction at the proximal end of the invention.

### DETAILED DESCRIPTION OF THE INVENTION

The present invention is directed to a cleaning device which mechanically clears stents which have become occluded with biological material such as biofilm and/or biliary sludge. The cleaning device of the invention comprises a rotating brush mounted on the distal end of a shaft which is disposed within a flexible, elongated tubular outer sheath, such as a catheter, that can be inserted through the working lumen of an endoscope or similar surgical device. In alternative embodiments, the device of the invention can also have provisions for fluid irrigation of the stent and for vacuum removal of debris resulting from the cleaning operation. The invention further encompasses a method of clearing occluded stents and for inhibiting further occlusion of the stents by the introduction of antibiotics at the stent site.

Reference will now be made in detail to the preferred embodiments of the invention, examples of which are illustrated in the accompanying drawings.

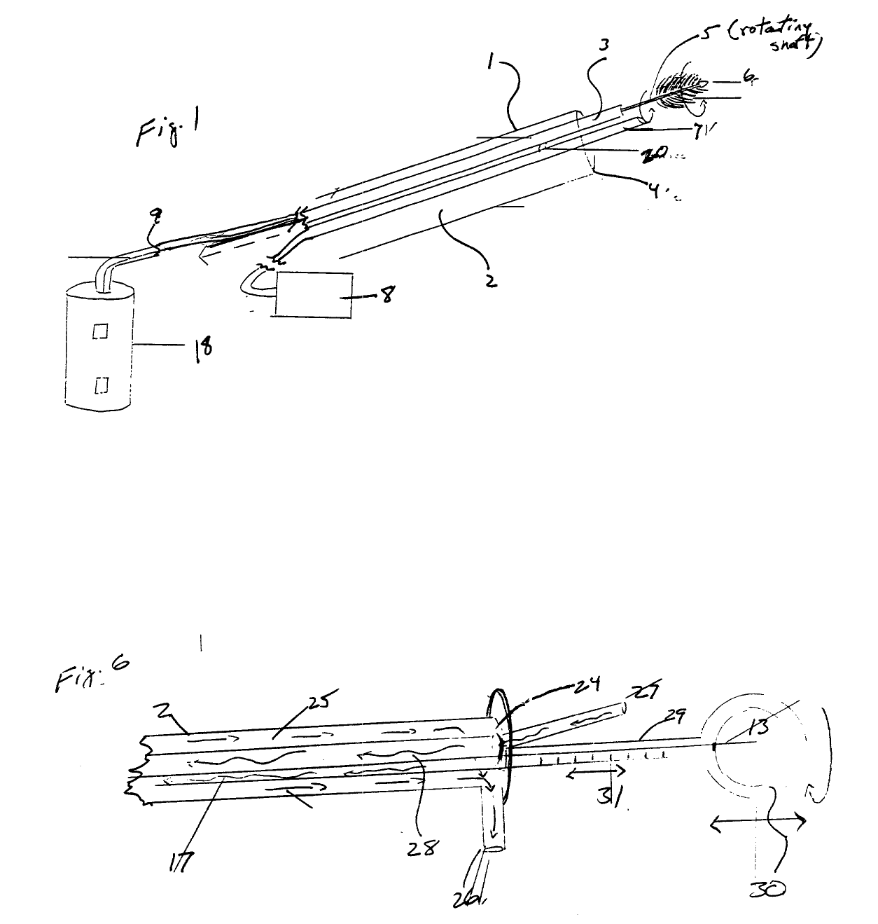

Directing attention to the embodiment of the present invention illustrated in FIG. 1 of the drawings, cleaning device 1 is shown having a flexible elongated tubular outer sheath or catheter tube 2 suitable for insertion into a working lumen of an endoscope. Disposed within catheter tube 2 is a non-rotating elongated tubular element 3 containing a rotating shaft 5 which terminates in a brush head 6 which may have a rigid, pointed tip to facilitate penetration of a stent (not shown). The bristles of the brush advantageously are relatively soft and non-rigid so that they collapse to facilitate removal of dislodged material from the stent and will not damage tissue or the stent. The bristles should, however, have enough rigidity to disrupt viscous biofilm.

A vacuum canula 7 is disposed within sheath 2, which is a catheter, alongside non-rotating tubular element 3 and exterids to a distal position beyond distal end 4 of sheath 2 and proximate brush head 6. Vacuum canula 7 is connected to tubular element 3 to maintain it in fixed position and extends through sheath 2 to connect at its proximal end to an external suction source 8. Rotating shaft 5, on which brush head 6 is mounted at the distal end of the device 1, is connected at its proximal end to a rotating line 9 driven by an external rotating drum 18. Rotating drum 18 may be hand-held and can rotate either electrically or manually in either the clockwise or counterclockwise direction at a controllable speed to drive the rotating brush head 6. To clean an occluded stent, the catheter 2 and brush head 6 are advanced through the lumen of an endoscope to the site of the stent where the rotating brush head 6 is gradually inserted into the stent. As it advances into the stent, the rotating action of the brush head 6 loosens accumulated biological material which is removed by suction through vacuum canula 7.

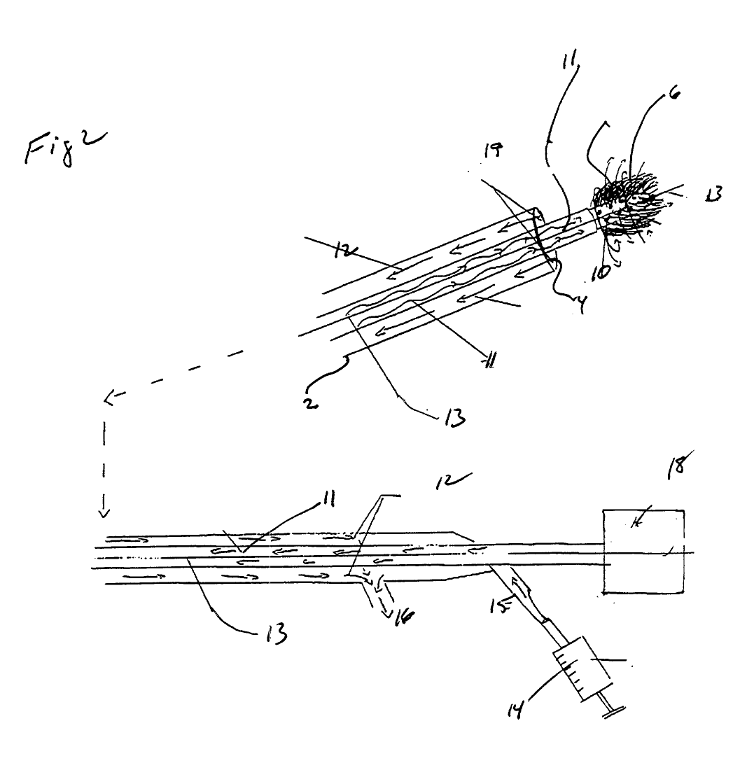

An alternative embodiment of the system of the invention is illustrated in FIG. 2 of the drawings in which the rotating shaft 11, on which brush head 6 is mounted, is hollow to allow fluid passage of an irrigating solution. One or more holes 10 are provided in brush head 6 to permit the irrigating solution to be discharged into the area of the brush head 6 to perform its cleaning function. Debris which is loosened and removed by this cleaning action, along with irrigating solution, is withdrawn through vacuum holes 19 in the distal end 4 of the outer sheath 2 by a vacuum applied to channel 12 formed between the inner rotating shaft 11 and the inner walls of the outer sheath 2. The evacuating vacuum is supplied to vacuum channel 12 through vacuum outlet 16 which is connected to a vacuum source (not shown). Irrigation solution, which may or may not contain an antibiotic, is conveniently applied by means of a pump, syringe, or other supply device 14 to infusion port 15 and into inner rotating shaft 11 so that it ultimately emerges from holes 10 in the rotating brush head 6 at the site of the stent. To facilitate accurate placement of brush head 6 into a stent, a guide wire 13 is provided coaxially with shaft 11 and the head of brush 6. The guide wire 13 is extended through the end of the brush 6 and can be extended well beyond the brush 6 to be prepositioned into and through the stent so that the cleaning brush 6 can then be advanced over the guide wire 13 accurately to that site. Once in position, the guide wire 13 may be withdrawn through the proximal end of the device either before or after the cleaning procedure is completed.

FIG. 3 of the drawings illustrates an embodiment of the invention in which a hollow, inner sheath 17 is provided concentrically with shaft 21 of the brush head 6 and within the outer sheath 2 to supply a vacuum at the distal end of sheath 17 through holes 19 to remove abraded debris from the cleaning site. Brush head 6 is mounted on the end of rotating shaft 21 just beyond the end 22 of inner sheath 17 and holes 19.

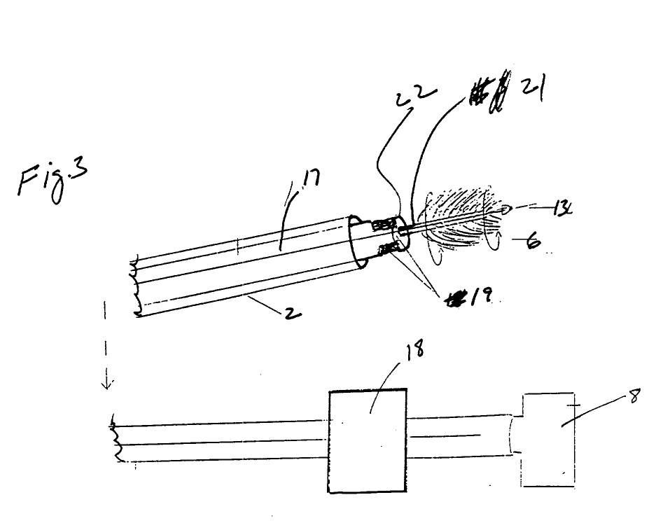

In the embodiment of the invention illustrated in FIG. 4, the hollow inner sheath 17 extends beyond distal end 23 of outer sheath 2 and becomes the rotating shaft for brush head 6. Brush head 6 has holes 10 in it for passage of irrigating solution fed through the hollow inner sheath 17 from a syringe 14 disposed at the proximal end of the device. As with the other illustrated embodiments of the invention, rotation is imparted to sheath 17 by rotational drum 18 at the proximal end of the unit, and a guide wire 13 is coaxially deployed within the inner sheath 17 to assist in positioning the device for cleaning stents.

The entire cleaning system of the invention is contained within a sheath or catheter that can be inserted through the working lumen of an endoscope, laparoscope, or similar medical device, or may be inserted percutaneously. To facilitate movement of the catheter, the catheter and its component elements can be provided with a slippery coating which can be a lubricious or hydrophilic substance such as hydrogel or silicone. Additionally, the catheter can be constructed of, or coated with, polytetrafluoroethylene or polyurethane.

The brush and the tip of the vacuum canula extend out through the distal end of the catheter but can be retracted into the catheter before positioning the device inside the stent. Distal retraction has the advantage of creating a low profile configuration that is easier to insert through a narrow lumen or tight junction, such as the sphincter of oddi.

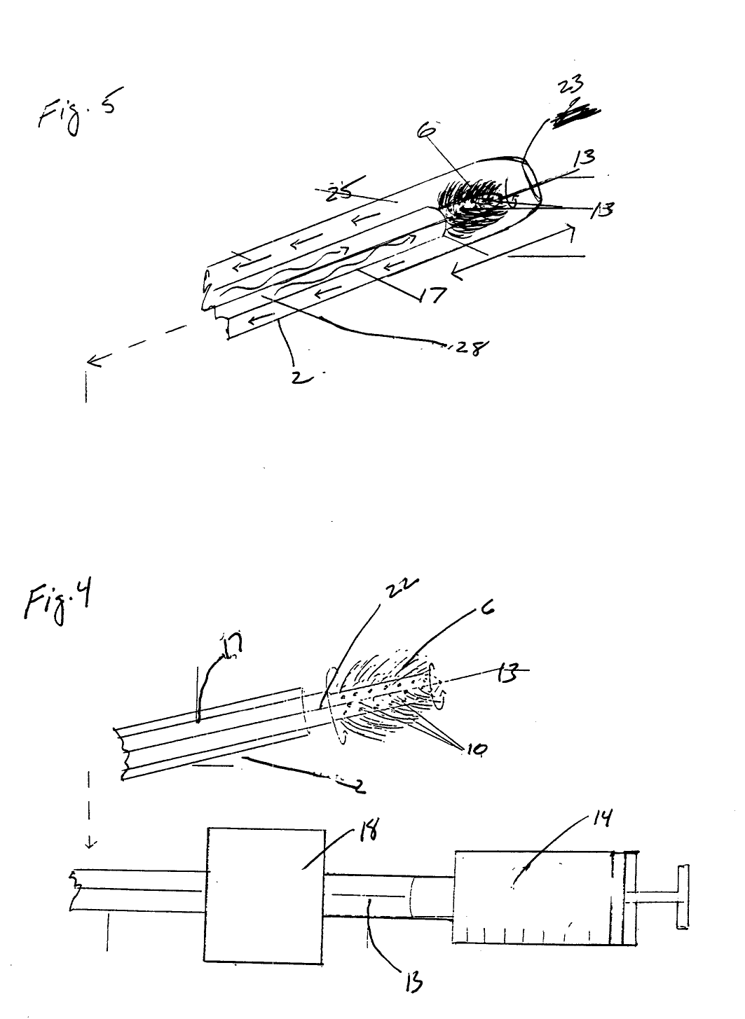

As shown in FIGS. 5 and 6, brush head 6 is retracted within the distal end 23 of outer sheath 2. Retraction/protraction of the brush head 6 is controlled by rotatable, sliding member 29 attached to the proximal end of inner sheath 17 along with infusion port 27 for introduction of irrigating fluid into inner channel 28. As previously noted, a guidewire 13 can also extend through channel 28, emerging at the tip of brush head 6. Spent irrigating solution and debris from the stent site is removed through vacuum channel 25 exiting at vacuum port 26. Conveniently, a rotatable handle 30 is provided at the end of sliding member 29 with markers 31 to gauge the extent of retraction/protraction of brush head 6.

The brush can be a single use appliance that can be disconnected from the reusable external rotating system. The vacuum canula can also be disposable.

As heretofore noted, in accordance with the method of the invention, occluded stents are mechanically cleared of occluding material and further occlusion inhibited by introducing the rotatable brush head of the device of the invention into the locus of the implanted and occluded stent and then advancing the brush head into the interior of the stent while the brush head rotates to abrade and remove occluding material. Placement of the rotatable brush head at the locus of the stent is facilitated by first passing a guidewire through the body tract into and through the stent and then threading the rotatable brush and shaft over the guidewire and advancing them until the brush head is positioned at the locus of the stent. Removal of abraded material from the stent is also facilitated by introduction of a washing fluid proximate the brush head, either during or subsequent to actual abrading of the material with the rotating brush. Both the washing fluid and the abraded material can be removed from the locus of the stent by suction through a vacuum line in the device.

It is particularly useful to employ the cleaning device of the invention in combination with an endoscope. According to this procedure, an endoscope having at least one working channel is introduced into the internal body passage and advanced as far as possible toward the implanted, occluded stent. Often, because of the size of the endoscope it cannot be advanced fully to the actual locus of the stent. A guidewire can then be inserted into the working channel of the endoscope and advanced through the endoscope to the stent. If the occlusion of the interior of the stent is not complete, the guidewire can be passed all the way through the stent. With the distal end of the guidewire positioned either in or through the stent, the axial channel running through the cleaning device of the invention is positioned over the proximal end of the guidewire, and the entire device advanced over the guidewire through the working lumen of the endoscope to the stent where the clearing procedure proceeds as heretofore described.

The cleaning system of the invention can also be employed with medical devices other than endoscopes. Alternative procedures from that described above can be used to deploy the cleaning system of the invention to clean occluded stents. For example, once the end of the guide wire is in place at the locus of the stent, the endoscope used to optically track and position the wire can be removed and the brush passed over the guidewire into the stent. Proper positioning of the brush can also be accomplished by providing the brush with a radiopaque marker to permit tracking with fluoroscopic guidance as the catheter is passed into the body toward the stent.

It may also be advantageous to employ the cleaning modes of the invention sequentially rather than simultaneously and, generally, the irrigation and suction modes are not employed simultaneously. That is, initial disrupting of the biofilm in the stent with the inserted rotating brush is followed by irrigation, then vacuum removal of debris and, finally, irrigation with an antibiotic solution.

The invention permits antibiotics at high concentration to be introduced directly into the site of cleaning. Further, the mechanical disruption of biofilm by the action of the brush in the stent greatly increases the susceptibility of the bacteria to the effect of the antibiotic solution thereby aiding in preventing future occlusion of the stent. An appropriate solution contains a broad-spectrum antibiotic, such as ciprofloxacin, combined with a biofilm inhibitor. Ciprofloxacin is a powerful, broad spectrum antibiotic that is well suited to biliary and urinary tract infections, especially when used in conjunction with mechanical disruption in accordance with the present invention.

In addition to a broad-spectrum antibiotic, salicylic acid or bismuth subsalicylate may be advantageously added to the irrigation solution. Salicylates have an inhibitory effect on bacterial polysaccharide production and on initial bacterial adherence. They are also known to potentiate the actions of some antibiotics, such as amino glycosides. In addition, salicylic acid is able to reduce mucin secretions, which further protects biofilm from antibiotics and contributes to biliary sludge. Furthermore, the bismuth moiety of bismuth subsalicylate decreases bacterial viability, which bolsters the effect of an antibiotic even further.

While salicylates enhance the activity of some antibiotics, they drastically diminish the antimicrobial activity of most others. Salicylates act as antagonists by repressing the synthesis of porin channels in bacteria, thereby restricting antibiotic transport through the outer membrane. Therefore, salicylates should not be combined with agents that rely on porin channel access, such as most β-lactam antibiotics, tetracyclines, sulfonamides, clindamycin, norfloxacin. Bismuth subsalicylate does not decrease ciprofloxacin bioavailability.

It may also be desirable to irrigate urethral stents with solutions that dissolve urinary encrustations, such as Albright's citric acid solution, Renacidin, or Suby's G and M solutions. These solutions may be combined with or followed by an antibiotic solution wash.

Irrigating the stent and/or biliary tract with an antimicrobial solution while mechanically disrupting the biofilm also increases the amount of bacteria that can be removed. In addition, it can prevent the adherence and growth of new bacteria for a longer period of time, thereby extending the patency rate of the prosthesis.

In addition to using this irrigating system for clearing an occluded stent, it can also be used as a prophylactic measure. For example, any time the biliary or urinary tracts are invaded endoscopically, these normally sterile environments become contaminated with foreign bacteria. Irrigating these tracts with an antibacterial solution prior to stent implantation, and again after implantation, can help reduce bacterial adherence to the stent and mucosa. Once the intruding microbes are eliminated, the normal flow of bile or urine should continue to flush the system! of bacteria. Also, if a sphincterotomy is performed before stent implantation, irrigating the system may help accelerate the healing process.

Systemically re-coating a medical device in situ can be costly or even toxic and as explained earlier, the concentration of antibiotics secreted in bile decreases with biliary obstruction, so orally coating a biliary stent is difficult in an occluded system. This invention can be used to directly re-coat stent materials after they have become depleted. The rotating brush can be used to clean the surface of the device and the irrigating system used to introduce a secondary coating.

It will be apparent to those skilled in the art that various modifications and variations can be made in the device and method of the present invention without departing from the spirit or scope of the invention. Thus, it is intended that the present invention cover the modifications and variations of this invention provided they can come within the scope of the appended claims and their equivalents.

What is claimed is:

1. A surgical cleaning device comprising:
an elongated tubular outer sheath;
a rotatable shaft having a distal end, the shaft being axially disposed in said outer sheath; and
a brush attached to said distal end of the shaft.

2. The surgical device of claim 1 wherein said brush has an interior compartment and said rotatable shaft is hollow and defines a flow path therethrough in fluid communication with said interior compartment of said brush, the brush having one or more exit apertures therein for dispensing fluid from said compartment.

3. The surgical cleaning device of claim 1 wherein the space between the interior of the outer sheath and said rotatable shaft defines an elongated channel terminating in one or more apertures in the distal end of said sheath proximate said brush, said channel having a proximal end for attachment to a vacuum source to apply suction to said channel.

4. The surgical cleaning device of claim 1 wherein an elongated channel extends concentrically through said rotatable shaft to accommodate a guide wire.

5. The surgical cleaning device of claim 1 wherein said rotatable shaft is concentrically disposed within a non-rotating elongated hollow shaft disposed within said sheath.

6. The surgical cleaning device of claim 5 wherein one or more additional non-rotatable elongated hollow tubular members are provided extending parallel to said rotatable shaft within said outer sheath and having distal terminus proximate said brush.

7. The surgical cleaning device of claim 6 wherein at least one of said additional tubular members is connected at its proximal end to a vacuum source.

8. The surgical cleaning device of claim 5 wherein said non-rotatable shaft is a vacuum line whose distal end terminates in one or more apertures proximate said brush.

9. The surgical cleaning device of claim 3 wherein said rotatable shaft is hollow and defines a flow path which is in fluid communication with an interior compartment of said brush, said brush having a plurality of exit apertures therein for dispensing fluids.

10. A surgical device for cleaning implanted stents which comprises: a flexible tubular outer sheath; a non-rotatable hollow shaft disposed axially within said sheath and disposed within said hollow shaft a rotatable first shaft whose distal end is terminally attached to a brush rotatable within said hollow shaft; tubular channel disposed concentrically within said rotatable shaft to accommodate a guidewire.

11. The surgical device of claim 10 wherein a second non-rotatable shaft is disposed within said sheath and extends parallel to the first shaft; said second non-rotating shaft being adapted for attachment at its proximal end to a vacuum source and terminating in one or more apertures at its distal end proximate said brush.

12. The surgical device of claim 10 wherein said non-rotatable shaft is adapted for attachment at its proximal end to a vacuum source and terminates in one or more apertures at its distal end proximate said brush.

13. The surgical device of claim 10 wherein said brush has a conical configuration with substantially flexible bristles.

14. A surgical device for cleaning implanted stents which comprises: a flexible tubular outer sheath; a rotatable hollow shaft disposed axially within said sheath having a distal end; a brush rotatable with said shaft and attached to the distal end thereof, said hollow shaft defining a flow path for fluids and being in fluid communication with an interior compartment of said brush, said brush having one or more apertures therein for dispensing fluid; and a tubular channel disposed axially within said rotatable hollow shaft to accommodate a guidewire.

15. The surgical device of claim 14 wherein the space between the interior of the outer sheath and the rotatable shaft defines an elongated vacuum channel which terminates in one or more apertures in the distal end of the sheath proximate said brush, and has a proximal end for attachment to a vacuum source for applying suction through the vacuum channel proximate to the brush.

16. The surgical device of claim 14 wherein said brush has a conical configuration with substantially flexible bristles.

17. The surgical device of claim 10 wherein the proximal end of said rotatable shaft is attached to means for imparting rotation to said shaft.

18. The surgical device of claim 14 wherein the proximal end of said rotatable shaft is attached to means for imparting rotation to said shaft.

19. A method for clearing an occluded, implanted stent and inhibiting further occlusion thereof, comprising the steps of:
introducing into the locus of an occluded implanted stent a surgical cleaning device comprising a catheter having a rotatable shaft disposed therein whose distal end is terminally attached to a brush which rotates with said shaft;
initially inserting at least a portion of the brush into the stent; and
rotating said brush as it is advanced into the stent to thereby abrade the occluding material in the interior of the stent.

20. The method of claim 19 wherein said cleaning device is introduced into the locus of the stent through the lumen of an invasive medical scope.

21. The method of claim 19 wherein the distal end of a guidewire is first introduced into the locus of the stent, the surgical cleaning device is then positioned over the guidewire by means of a channel concentrically disposed in the shaft and brush, and the brush is then advanced over the guidewire to the distal terminus of the guidewire at the locus of the stent.

22. A method for clearing an occluded, implanted stent and inhibiting further occlusion thereof, comprising the steps of:
introducing into the locus of an occluded, implanted stent a surgical cleaning device comprising a catheter having disposed therein a rotatable shaft whose distal end is terminally attached to a brush which rotates with said shaft, and one or more fluid conduits also disposed in said catheter for providing irrigating fluid to said brush;
initially inserting at least a portion of the brush into the stent;
rotating said brush as it is advanced into the stent to thereby abrade the occluding material in the interior of the stent; and
irrigating the stent with fluid provided through said conduit to the brush.

23. The method of claim 22 wherein said irrigating fluid and material from the stent are removed from the locus of the stent through a vacuum line disposed in the catheter.

24. The method of claim 22 wherein the cleaning device is introduced into the locus of the stent through the lumen of an invasive medical scope.

25. The method of claim 22 wherein the irrigating solution contains one or more fluids selected from antibiotics, polysaccharide inhibitors, crystallization inhibitors, dissolutors, mucolytic agents and saline solution.

26. A method for clearing an occluded, implanted stent and inhibiting further occlusion thereof, comprising the steps of:
introducing through the working lumen of a medical scope into the locus of an occluded, implanted stent a surgical cleaning device comprising a catheter having disposed therein a rotatable shaft whose distal end is terminally attached to a brush which rotates with said shaft, and one or more fluid conduits also disposed in said catheter for providing irrigating fluid containing an antibiotic to said brush;
initially inserting at least a portion of the brush into the stent;
rotating said brush as it is advanced into the stent to thereby abrade the occluding material in the interior of the stent; and
irrigating the stent with said irrigating fluid containing an antibiotic provided through said conduit to the brush.

27. A method for clearing an occluded implanted stent and inhibiting further occlusion thereof, comprising the steps of:
introducing an endoscope into an internal body passage containing an occluded, implanted stent;
inserting a guidewire into a working lumen of said endoscope and passing the distal end of the guidewire through the lumen to the locus of the stent;
threading onto the guidewire a cleaning device having a through passage for the guidewire and comprising a catheter having disposed therein a rotatable shaft whose distal end is terminally attached to a brush which rotates with said shaft, and one or more fluid conduits also disposed in said catheter for providing irrigating fluid to said brush;
advancing said cleaning device over the guidewire until said brush reaches the locus of the stent;
initially inserting at least a portion of the brush into the stent;
rotating said stent as it is advanced into the stent to thereby abrade the occluding material in the interior of the stent;
irrigating the stent with fluid provided through said conduit to the brush; and
removing the abraded material and irrigating fluid from the locus of the stent through a vacuum line disposed in the catheter and connected at its proximal end to a vacuum source.

28. The method of claim 27 wherein said irrigating fluid contains one or more fluids selected from antibiotics, polysaccharide inhibitors, crystallization inhibitors, dissolutors, mucolytic agents and saline solution.

29. The method of claim 27 wherein said step of rotating, irrigating and removing material and fluid are carried out sequentially.

30. The surgical device of claim 10 wherein the distal end of said rotatable shaft projects beyond said sheath, and is retractable therein.

31. The surgical device of claim 14 wherein the distal end of said rotatable shaft projects beyond said sheath, and is retractable therein.

32. The method of claim 20 wherein said medical scope is an endoscope or laparoscope.

33. The method of claim 24 wherein said medical scope is an endoscope or laparoscope.

34. The method of claim 26 wherein said medical scope is an endoscope or laparoscope.
